(19) United States
(12) Patent Application Publication
(10) Pub. No.: US 2024/0043931 A1
(43) Pub. Date: Feb. 8, 2024

(54) MULTI-BIOMARKER ASSAY TO ASSESS MULTIPLE SCLEROSIS DISEASE ACTIVITY

(71) Applicants: UNIVERSITY OF MARYLAND, BALTIMORE, Baltimore, MD (US); The United States Government as represented by the Department of Veterans Affairs, Washington, DC (US)

(72) Inventors: Horea G. Rus, Catonsville, MD (US); Freidrich ANSELMO, Germantown, MD (US); Alexandru TATOMIR, Baltimore, MD (US)

(73) Assignees: UNIVERSITY OF MARYLAND, BALTIMORE, Baltimore, MD (US); The United States Government as represented by the Department of Veterans Affairs, Washington, DC (US)

(21) Appl. No.: 18/364,581

(22) Filed: Aug. 3, 2023

Related U.S. Application Data
(60) Provisional application No. 63/394,662, filed on Aug. 3, 2022.

(51) Int. Cl. C12Q 1/6883; G01N 33/68

(52) U.S. Cl. CPC C12Q 1/6883; G01N 33/6896; C12Q 2600/112; G01N 2800/60; G01N 2800/52; G01N 2800/285; C12Q 2600/106; C12Q 2600/158

(57) ABSTRACT

A MS disease activity (MSDA) score is provided as a tool to detect relapse and response to glatiramer acetate (GA) therapy in subjects having relapsing-remitting multiple sclerosis (RRMS) using the blood-based biomarkers SIRT1, RGC-32, FasL, IL-21, pSIRT1 and JNK p54.

### STATEMENT OF FEDERALLY SPONSORED RESEARCH AND DEVELOPMENT

This invention was made with government support under VA Merit Review Grant Number 101BX001458 awarded by the United States Department of Veterans Affairs and under Grant Number NS042011 awarded by the National Institutes of Health. The government has certain rights in the invention.

### SEQUENCE LISTING

A sequence listing in electronic (XML file) format is filed with this application and incorporated herein by reference. The name of the ASCII text file is “2023-1121A.xml”; the file was created on Aug. 1, 2023; the size of the file is 18,598 bytes.

### BACKGROUND OF INVENTION

Multiple sclerosis (MS) is a chronic, autoimmune, demyelinating disease characterized by chronic inflammation of the central nervous system in which many factors (genetic and environmental) may act together to influence disease susceptibility and progression (Frohman et al., 2006; Keegan and Noseworthy, 2002). While a large body of work has enhanced an understanding of the fundamental nature of MS, basic research into its etiology, pathophysiology, and treatment faces enormous challenges, and this may in part be due to the great variability in the clinical presentation and course of MS (Compston and Coles, 2008; Frohman et al., 2006; Keegan and Noseworthy, 2002).

Relapsing-remitting multiple sclerosis (RRMS) is one of the two main forms of the disease, the other being primary-progressive multiple sclerosis (PPMS). RRMS is characterized by periods of worsening neurologic function (Lublin et al., 2014; Polman et al., 2011). These periods, termed relapses or flare-ups, are followed by partial or complete recovery periods (remissions), during which symptoms improve partially or completely, and there is no apparent disease progression. RRMS is the most common disease course at the time of diagnosis (Lublin et al., 2014; Polman et al., 2011). Approximately 85 percent of people are initially diagnosed with RRMS, compared to 10-15 percent with progressive forms of the disease (Lublin et al., 2014; Polman et al., 2011). RRMS is defined by inflammatory attacks on myelin, as well as the nerve fibers themselves (Lublin et al., 2014; Polman et al., 2011). During these inflammatory attacks, activated immune cells cause small, localized areas of damage which produce the symptoms of MS (Lublin et al., 2014; Polman et al., 2011). Because the location of the damage is so variable, no two people have exactly the same symptoms.

While RRMS is defined by attacks of inflammation (relapses) in the central nervous system (CNS), the progressive form of MS involves much less of this type of inflammation. Subjects with RRMS tend to have more brain lesions, also called plaques or scars, detectable via magnetic resonance imaging (MRI) scans, and these lesions contain more inflammatory cells. Subjects with PPMS tend to have more spinal cord lesions, which contain fewer inflammatory cells.

A subject is diagnosed as having RRMS when test results provide evidence of at least two separate areas of damage to the myelin in the CNS that have occurred at different points in time (nationalmssociety.org). Tests that may be used to determine whether there is relevant damage to myelin in a subject include magnetic resonance imaging (MM), visual evoked potential (VEP) testing, and analysis of the cerebrospinal fluid (nationalmssociety.org).

These tests have drawbacks, including pain, risks and costs. For example, a brain MRI is an expensive test that is hard to tolerate by claustrophobic patients. In addition, identification of active lesions on a brain MRI requires the administration of the gadolinium to a subject, which poses significant risks of allergic reactions and gadolinium-associated nephrogenic systemic fibrosis (Grobner T., 2006; Hellman, R., 2011). Indeed, gadolinium associated nephrogenic systemic fibrosis is a systemic, sometimes fatal (mortality rate up to 30%), disabling disease, mimicking scleroderma (Grobner T., 2006; Hellman, R., 2011).

Quantitative and regular assessment of disease activity in MS is required to achieve treatment targets such as remission and to optimize clinical outcomes. To predict relapses and monitor treatment response, a measure of disease activity in MS should reflect the pathological processes resulting in brain tissue damage and functional disability.

The development of additional means for predicting relapses and monitoring treatment response in a subject as having RRMS will greatly aid clinicians in quickly and accurately prescribing appropriate treatment. The present invention is directed to these and other important goals.

### BRIEF SUMMARY OF INVENTION

The present invention is generally directed to a MS Disease Activity (MSDA) score for use as a tool in detecting disease relapse and detecting response to glatiramer acetate (GA) therapy in subjects having relapsing-remitting multiple sclerosis (RRMS). As described herein, the MSDA test was developed using the blood-based biomarkers SIRT1, RGC-32, FasL, IL-21, pSIRT1 and JNK p54. The resulting MSDA score can be used in determining whether a subject previously diagnosed as having RRMS is undergoing a relapse of the disease and in predicting whether a subject having RRMS will respond to treatment, such as GA treatment.

As discussed in detail below, the inventors studied the expression of several biomarkers, including SIRT1, RGC-32, FasL, IL-21, pSIRT1 and JNK p54 in RRMS patients undergoing relapse (relapse RRMS) and compared expression levels to those of healthy controls and RRMS patients not experiencing relapse (stable RRMS). Levels of SIRT1, RGC-32 and FasL mRNA were found to be significantly reduced in RRMS patients with relapse as compared to control patients, while levels of IL-21 mRNA were found to be increased. Similarly, levels of pSIRT1 protein were found to be significantly reduced in RRMS patients with relapse as compared to control patients, while levels of JNK p54 protein were found to be increased.

Thus, it was found that changes in the expression levels of these six molecules could be used as markers of disease activity in patients with RRMS.

In a first embodiment, the invention is generally drawn to methods for determining whether a subject having relapsing-remitting multiple sclerosis (RRMS) is undergoing relapse of the disease by monitoring levels of mRNA and protein expression of certain biomarkers. In particular, the invention is drawn to methods for determining whether a subject having relapsing-remitting multiple sclerosis (RRMS) is undergoing relapse of the disease, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <3.05, a value of +1 is assigned, and when the SIRT1/mRNA standard ratio is ≥3.05, a value of −1 is assigned,
wherein when a ratio is calculated for RGC-32 and the RGC-32/mRNA standard ratio is <1.27, a value of +1 is assigned, and when the RGC-32/mRNA standard ratio is ≥1.27, a value of −1 is assigned,
wherein when a ratio is calculated for FasL and the FasL/mRNA standard ratio is <52.6, a value of +1 is assigned, and when the FasL/mRNA standard ratio is ≥52.6, a value of −1 is assigned,
wherein when a ratio is calculated for IL-21 and the IL-21/mRNA standard ratio is >16.9, a value of +1 is assigned, and when the IL-21/mRNA standard ratio is ≤16.9, a value of −1 is assigned,
wherein when a ratio is calculated for pSIRT1 and the pSIRT1/protein standard ratio is <3.05, a value of +1 is assigned, and when the pSIRT1/protein standard ratio is ≥3.05, a value of −1 is assigned,
wherein when a ratio is calculated for JNK p54 and the JNK p54/protein standard ratio is >1.2, a value of +1 is assigned, and when the JNK p54/protein standard ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In an exemplary aspect of the first embodiment, the invention is drawn to methods for determining whether a subject having relapsing-remitting multiple sclerosis (RRMS) is undergoing relapse of the disease, comprising
(i) determining mRNA expression levels for one or more first biomarkers selected from the group consisting of SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each selected first biomarker;
(iv) determining protein expression levels for one or more second biomarkers selected from the group consisting of pSIRT1 and JNK p54 in a second population of cells,
(v) determining protein expression levels for β-actin in the same second population of cells, and
(vi) calculating a ratio of second biomarker protein expression to β-actin expression for each selected second biomarker;
wherein when a ratio is calculated for SIRT1 and the SIRT1/L13 mRNA ratio is <3.05, a value of +1 is assigned, and when the SIRT1/L13 mRNA ratio is ≥3.05, a value of −1 is assigned,
wherein when a ratio is calculated for RGC-32 and the RGC-32/L13 mRNA ratio is <1.27, a value of +1 is assigned, and when the RGC-32/L13 mRNA ratio is ≥1.27, a value of −1 is assigned,
wherein when a ratio is calculated for FasL and the FasL/L13 mRNA ratio is <52.6, a value of +1 is assigned, and when the FasL/L13 mRNA ratio is ≥52.6, a value of −1 is assigned,
wherein when a ratio is calculated for IL-21 and the IL-21/L13 mRNA ratio is >16.9, a value of +1 is assigned, and when the IL-21/L13 mRNA ratio is ≤16.9, a value of −1 is assigned,
wherein when a ratio is calculated for pSIRT1 and the pSIRT1/β-actin ratio is <3.05, a value of +1 is assigned, and when the pSIRT1/β-actin ratio is ≥3.05, a value of −1 is assigned,
wherein when a ratio is calculated for JNK p54 and the JNK p54/β-actin ratio is >1.2, a value of +1 is assigned, and when the JNK p54/β-actin ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In a further exemplary aspect of the first embodiment, the invention is drawn to methods for determining whether a subject having relapsing-remitting multiple sclerosis (RRMS) is undergoing relapse of the disease, comprising
(i) determining mRNA expression levels for first biomarkers SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each first biomarker;
(iv) determining protein expression levels for second biomarkers pSIRT1 and JNK p54 in a second population of cells,
(v) determining protein expression levels for a selected protein standard in the same second population of cells, and
(vi) calculating a ratio of second biomarker protein expression to protein standard expression for each second biomarker;
wherein when the ratio calculated for SIRT1 and the SIRT1/mRNA standard ratio is <3.05, a value of +1 is assigned, and when the SIRT1/mRNA standard ratio is ≥3.05, a value of −1 is assigned,
wherein when the ratio calculated for RGC-32 and the RGC-32/mRNA standard ratio is <1.27, a value of +1 is assigned, and when the RGC-32/mRNA standard ratio is ≥1.27, a value of −1 is assigned,
wherein when the ratio calculated for FasL and the FasL/mRNA standard ratio is <52.6, a value of +1 is assigned, and when the FasL/standard mRNA standard ratio is ≥52.6, a value of −1 is assigned,
wherein when the ratio calculated for IL-21 and the IL-21/mRNA standard ratio is >16.9, a value of +1 is assigned, and when the IL-21/mRNA standard ratio is ≤16.9, a value of −1 is assigned,
wherein when the ratio calculated for pSIRT1 and the pSIRT1/protein standard ratio is <3.05, a value of +1 is assigned, and when the pSIRT1/protein standard ratio is ≥3.05, a value of −1 is assigned,
wherein when the ratio calculated for JNK p54 and the JNK p54/protein standard ratio is >1.2, a value of +1 is assigned, and when the JNK p54/protein standard ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In yet a further exemplary aspect of the first embodiment, the invention is drawn to methods for determining whether a subject having relapsing-remitting multiple sclerosis (RRMS) is undergoing relapse of the disease, comprising
(i) determining mRNA expression levels for first biomarkers SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each first biomarker;
(iv) determining protein expression levels for second biomarkers pSIRT1 and JNK p54 in a second population of cells,
(v) determining protein expression levels for β-actin in the same second population of cells, and
(vi) calculating a ratio of second biomarker protein expression to β-actin expression for each second biomarker;
wherein when the ratio calculated for SIRT1 and the SIRT1/L13 mRNA ratio is <3.05, a value of +1 is assigned, and when the SIRT1/L13 mRNA ratio is ≥3.05, a value of −1 is assigned,
wherein when the ratio calculated for RGC-32 and the RGC-32/L13 mRNA ratio is <1.27, a value of +1 is assigned, and when the RGC-32/L13 mRNA ratio is ≥1.27, a value of −1 is assigned,
wherein when the ratio calculated for FasL and the FasL/L13 mRNA ratio is <52.6, a value of +1 is assigned, and when the FasL/L13 mRNA ratio is ≥52.6, a value of −1 is assigned,
wherein when the ratio calculated for IL-21 and the IL-21/L13 mRNA ratio is >16.9, a value of +1 is assigned, and when the IL-21/L13 mRNA ratio is ≤16.9, a value of −1 is assigned,
wherein when the ratio calculated for pSIRT1 and the pSIRT1/β-actin ratio is <3.05, a value of +1 is assigned, and when the pSIRT1/β-actin ratio is ≥3.05, a value of −1 is assigned,
wherein when the ratio calculated for JNK p54 and the JNK p54/β-actin ratio is >1.2, a value of +1 is assigned, and when the JNK p54/β-actin ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In aspects of this embodiment, when the calculated sum of assigned values is −1 to +1, the results are indeterminate.

In relevant aspects of this embodiment, the mRNA standard may be, but is not limited to, L13 mRNA, beta-actin MRNA, glyceraldehyde-3-phosphate dehydrogenase (GAPH) mRNA, and ribosome small subunit (18S) mRNA. In selected aspects of this embodiment, the mRNA standard is L13 mRNA.

In relevant aspects of this embodiment, the protein standard may be, but is not limited to, β-actin, β-tubulin and GAPDH. In selected aspects of this embodiment, the protein standard is β-actin.

In relevant aspects of this embodiment, mRNA expression levels for one, two, three or all of the first biomarkers is determined.

In relevant aspects of this embodiment, protein expression levels for one or both of the second biomarkers is determined.

In relevant aspects of this embodiment, mRNA expression levels for each of the first biomarkers are determined and protein expression levels for both of the second biomarkers are determined.

In all aspects of this embodiment, the method further comprises administering a therapeutically-effective amount of a treatment for RRMS to the subject when the subject is determined to be undergoing relapse. Suitable treatments include, but are not limited to, glatiramer acetate (GA), beta-interferons, teriflunomide, fingolimod, dimethyl fumarate, natalizumab, ozanimod, ponesimod, ocrelizumab and ofatumumab.

In a second embodiment, the invention is generally drawn to methods for determining whether a subject having relapsing-remitting multiple sclerosis (RRMS) will respond to a selected treatment, such as glatiramer acetate (GA). In particular, the invention is drawn to methods for determining whether a subject having relapsing-remitting multiple sclerosis (RRMS) will respond to treatment with GA, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <4.33, a value of +1 is assigned, and when the SIRT1/mRNA standard ratio is ≥4.33, a value of −1 is assigned,
wherein when a ratio is calculated for RGC-32 and the RGC-32/mRNA standard ratio is <2.52, a value of +1 is assigned, and when the RGC-32/mRNA standard ratio is ≥2.52, a value of −1 is assigned,
wherein when a ratio is calculated for FasL and the FasL/mRNA standard ratio is <85.4, a value of +1 is assigned, and when the FasL/mRNA standard ratio is ≥85.4, a value of −1 is assigned,
wherein when a ratio is calculated for IL-21 and the IL-21/mRNA standard ratio is >11.9, a value of +1 is assigned, and when the IL-21/mRNA standard ratio is ≤11.9, a value of −1 is assigned,
wherein when a ratio is calculated for pSIRT1 and the pSIRT1/protein standard ratio is <0.3, a value of +1 is assigned, and when the pSIRT1/protein standard ratio is ≥0.3, a value of −1 is assigned,
wherein when a ratio is calculated for JNK p54 and the JNK p54/protein standard ratio is >1.3, a value of +1 is assigned, and when the JNK p54/protein standard ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In an exemplary aspect of the second embodiment, the invention is drawn to methods for determining whether a subject having relapsing-remitting multiple sclerosis (RRMS) will respond to treatment with GA, comprising
(i) determining mRNA expression levels for one or more first biomarkers selected from the group consisting of SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each selected first biomarker;
(iv) determining protein expression levels for one or more second biomarkers selected from the group consisting of pSIRT1 and JNK p54 in a second population of cells, (v) determining protein expression levels for β-actin in the same second population of cells, and
(vi) calculating a ratio of second biomarker protein expression to β-actin expression for each selected second biomarker;
wherein when a ratio is calculated for SIRT1 and the SIRT1/L13 mRNA ratio is <4.33, a value of +1 is assigned, and when the SIRT1/L13 mRNA ratio is ≥4.33, a value of −1 is assigned,
wherein when a ratio is calculated for RGC-32 and the RGC-32/L13 mRNA ratio is <2.52, a value of +1 is assigned, and when the RGC-32/L13 mRNA ratio is ≥2.52, a value of −1 is assigned,
wherein when a ratio is calculated for FasL and the FasL/L13 mRNA ratio is <85.4, a value of +1 is assigned, and when the FasL/L13 mRNA ratio is ≥85.4, a value of −1 is assigned,
wherein when a ratio is calculated for IL-21 and the IL-21/L13 mRNA ratio is >11.9, a value of +1 is assigned, and when the IL-21/L13 mRNA ratio is ≤11.9, a value of −1 is assigned,
wherein when a ratio is calculated for pSIRT1 and the pSIRT1/β-actin ratio is <0.3, a value of +1 is assigned, and when the pSIRT1/β-actin ratio is ≥0.3, a value of −1 is assigned,
wherein when a ratio is calculated for JNK p54 and the JNK p54/β-actin ratio is >1.3, a value of +1 is assigned, and when the JNK p54/β-actin ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In a further exemplary aspect of the second embodiment, the invention is drawn to methods for determining whether a subject having relapsing-remitting multiple sclerosis (RRMS) will respond to treatment with GA, comprising
(i) determining mRNA expression levels for first biomarkers SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each first biomarker;
(iv) determining protein expression levels for second biomarkers pSIRT1 and JNK p54 in a second population of cells,
(v) determining protein expression levels for a selected protein standard in the same second population of cells, and
(vi) calculating a ratio of second biomarker protein expression to protein standard expression for each second biomarker;
wherein when the ratio calculated for SIRT1 and the SIRT1/mRNA standard ratio is <4.33, a value of +1 is assigned, and when the SIRT1/mRNA standard ratio is ≥4.33, a value of −1 is assigned,
wherein when the ratio calculated for RGC-32 and the RGC-32/mRNA standard ratio is <2.52, a value of +1 is assigned, and when the RGC-32/mRNA standard ratio is ≥2.52, a value of −1 is assigned,
wherein when the ratio calculated for FasL and the FasL/mRNA standard ratio is <85.4, a value of +1 is assigned, and when the FasL/mRNA standard ratio is ≥85.4, a value of −1 is assigned,
wherein when the ratio calculated for IL-21 and the IL-21/mRNA standard ratio is >11.9, a value of +1 is assigned, and when the IL-21/mRNA standard ratio is ≤11.9, a value of −1 is assigned,
wherein when the ratio calculated for pSIRT1 and the pSIRT1/protein standard ratio is <0.3, a value of +1 is assigned, and when the pSIRT1/protein standard ratio is ≥0.3, a value of −1 is assigned,
wherein when the ratio calculated for JNK p54 and the JNK p54/protein standard ratio is >1.3, a value of +1 is assigned, and when the JNK p54/protein standard ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In yet a further exemplary aspect of the second embodiment, the invention is drawn to methods for determining whether a subject having relapsing-remitting multiple sclerosis (RRMS) will respond to treatment with GA, comprising
(i) determining mRNA expression levels for first biomarkers SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each first biomarker;
(iv) determining protein expression levels for second biomarkers pSIRT1 and JNK p54 in a second population of cells,
(v) determining protein expression levels for β-actin in the same second population of cells, and
(vi) calculating a ratio of second biomarker protein expression to β-actin expression for each second biomarker;
wherein when the ratio calculated for SIRT1 and the SIRT1/L13 mRNA ratio is <4.33, a value of +1 is assigned, and when the SIRT1/L13 mRNA ratio is ≥4.33, a value of −1 is assigned,
wherein when the ratio calculated for RGC-32 and the RGC-32/L13 mRNA ratio is <2.52, a value of +1 is assigned, and when the RGC-32/L13 mRNA ratio is ≥2.52, a value of −1 is assigned,
wherein when the ratio calculated for FasL and the FasL/L13 mRNA ratio is <85.4, a value of +1 is assigned, and when the FasL/L13 mRNA ratio is ≥85.4, a value of −1 is assigned,
wherein when the ratio calculated for IL-21 and the IL-21/L13 mRNA ratio is >11.9, a value of +1 is assigned, and when the IL-21/L13 mRNA ratio is ≤11.9, a value of −1 is assigned,
wherein when the ratio calculated for pSIRT1 and the pSIRT1/β-actin ratio is <0.3, a value of +1 is assigned, and when the pSIRT1/β-actin ratio is ≥0.3, a value of −1 is assigned,
wherein when the ratio calculated for JNK p54 and the JNK p54/β-actin ratio is >1.3, a value of +1 is assigned, and when the JNK p54/β-actin ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In aspects of this embodiment, when the calculated sum of assigned values is −1 to +1, the results are indeterminate.

In relevant aspects of this embodiment, the mRNA standard may be, but is not limited to, L13 mRNA, beta-actin mRNA, glyceraldehyde-3-phosphate dehydrogenase (GAPDH) mRNA, and ribosome small subunit (18S) mRNA. In selected aspects of this embodiment, the mRNA standard is L13 mRNA.

In relevant aspects of this embodiment, the protein standard may be, but is not limited to, β-actin, β-tubulin and GAPDH. In selected aspects of this embodiment, the protein standard is β-actin.

In relevant aspects of this embodiment, mRNA expression levels for one, two, three or all of the first biomarkers is determined.

In relevant aspects of this embodiment, protein expression levels for one or both of the second biomarkers is determined.

In relevant aspects of this embodiment, mRNA expression levels for each of the first biomarkers are determined and protein expression levels for both of the second biomarkers are determined.

In relevant aspects of this embodiment, the method further comprises administering a therapeutically-effective amount of GA to the subject when the subject is determined to respond to treatment with GA.

In other relevant aspects of this embodiment, the method further comprises administering a therapeutically-effective amount of a non-GA treatment to the subject when the subject is determined not to respond to treatment with GA. Suitable treatments include, but are not limited to, beta-interferons, teriflunomide, fingolimod, dimethyl fumarate, natalizumab, ozanimod, ponesimod, ocrelizumab and ofatumumab.

In each of the embodiments and aspects of the invention, the population of cells may be, but is not limited to, peripheral blood mononuclear cells (PBMCs), CD4+ T cells, CD8+ T cells, MAB328+ cells, GFAP+ cells, leukocytes, monocytes, glial cells, and dendritic cells.

In each of the embodiments and aspects of the invention, mRNA expression may be determined using real-time polymerase chain reactions (RT-PCT).

In each of the embodiments and aspects of the invention, protein expression may be determined using Western blot analysis.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

FIG. 1. Selection of cut-off values for biomarkers used in the MSDA score. For each of the biomarkers used in the MSDA score, a cut-off value was obtained by using the receiver operating curve (ROC) analysis from data previously published (Ciriello et al., 2018; Anselmo et al., 2020; Kruszewski et al., 2015; Hewes et al., 2017) for both detecting relapses (A) and for detecting responses to GA (B).

FIG. 2. Generation of the MSDA score. Depending on the levels obtained for each of the six markers examined, a positive or negative value was assigned and used to establish a score of disease activity. By adding the values of all six biomarkers, an MSDA score was generated. An MSDA score above +1 indicated a relapse or non-response to GA treatment. An MSDA score less than −1 indicated that a patient was stable.

FIG. 3. MSDA Score reporting examples. A: An MSDA Score of −4 was found in this stable MS patient. B: An MSDA score of +2 was found in this MS patient with a relapse. C: A responder to GA treatment (MSDA score −4). D: A non-responder to GA patient (MSDA score +2).

FIG. 4. MSDA scores using the six biomarkers in MS patients included in the study. MSDA scores using the six biomarkers for the MS patients included in this study are shown in A. A statistically significant difference was found between the MSDA scores in stable vs. relapsing patients (p<0.0001). B. MSDA scores using the six biomarkers for the MS patients treated with GA. A statistically significant difference was found when comparing responders to GA treatment with and in non-responders to GA treatment (p=0.0001).

FIG. 5. Specificity and sensitivity of the MSDA score using the six biomarkers. Values for specificity, sensitivity, positive predictive value and negative predictive value are presented.

### DETAILED DESCRIPTION OF THE INVENTION

### I. Definitions

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. The Present Invention

While there is no cure for MS, recent advances in understanding MS pathology have led to the development of a plethora of disease modifying therapies (DMTs) that can successfully treat MS patients by intervening in key pathological processes (Trapp et al., 2008). Furthermore, quantitative and regular assessment of disease activity in MS is required to achieve treatment targets and optimize clinical outcomes, and therefore there is a significant need for the identification and validation of reliable biomarkers to predict MS relapse and response to therapy.

The goal of the present invention was to establish a MS Disease Activity (MSDA) score as a tool to detect relapses and response to therapeutics by using a panel of blood-based biomarkers in RRMS patients. As disclosed herein, the MSDA test measures six different biomarkers that have been associated with RRMS disease activity. As shown herein, these biomarkers are reliable indicators for predicting relapse as well as response to therapy (Tatomir et al., 2017; Ciriello et al., 2018; Anselmo et al., 2020). Since it measures six biomarkers, as opposed to measuring only one or two inflammatory markers such as oligoclonal bands or the IgG index in spinal fluid, this test is much more accurate than existing tests. It also has the advantage of using blood and therefore does not require spinal fluid. The MSDA score is designed to be used in individuals who have already been diagnosed with RRMS to assess their level of disease activity. It offers an objective and quantitative score beyond a physical exam that can be used to assess the degree of effectiveness of DMTs or biologics in controlling RRMS.

As briefly summarized above, the present inventors have found that the levels of the biomarkers SIRT1 (Sirtuin 1), RGC-32 (Response Gene to Complement-32), FasL (Fas ligand), IL-21, pSIRT1 (phosphorylated Sirtuin 1) and JNK (c-Jun N-terminal kinase) p54 vary in a subject having RRMS based on the whether the subject is experiencing a relapse or undergoing treatment. Among other observations, the inventors found SIRT1 levels were significantly reduced in MS patients with relapses as compared to control patients. In particular, decreased expression of SIRT1 was found in PBMCs during relapse, thus demonstrating the protein may be used as a marker of disease activity. These observations form the basis of the present invention which is generally directed to methods for determining whether a subject previously diagnosed as having MS is undergoing a relapse of the disease and predicting whether a subject having RRMS will respond to treatment with glatiramer acetate (GA) based on expression levels of the biomarkers SIRT1, RGC-32, FasL, IL-21, pSIRT1 and JNK p54.

In a first embodiment, the invention is thus generally drawn to methods for determining whether a subject having relapsing-remitting multiple sclerosis (RRMS) is undergoing relapse of the disease by monitoring levels of mRNA and protein expression of certain biomarkers. In particular, the invention is drawn to methods for determining whether a subject having relapsing-remitting multiple sclerosis (RRMS) is undergoing relapse of the disease, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <3.05, a value of +1 is assigned, and when the SIRT1/mRNA standard ratio is ≥3.05, a value of −1 is assigned,
wherein when a ratio is calculated for RGC-32 and the RGC-32/mRNA standard ratio is <1.27, a value of +1 is assigned, and when the RGC-32/mRNA standard ratio is ≥1.27, a value of −1 is assigned,
wherein when a ratio is calculated for FasL and the FasL/mRNA standard ratio is <52.6, a value of +1 is assigned, and when the FasL/mRNA standard ratio is ≥52.6, a value of −1 is assigned,
wherein when a ratio is calculated for IL-21 and the IL-21/mRNA standard ratio is >16.9, a value of +1 is assigned, and when the IL-21/mRNA standard ratio is ≤16.9, a value of −1 is assigned,
wherein when a ratio is calculated for pSIRT1 and the pSIRT1/protein standard ratio is <3.05, a value of +1 is assigned, and when the pSIRT1/protein standard ratio is ≥3.05, a value of −1 is assigned,
wherein when a ratio is calculated for JNK p54 and the JNK p54/protein standard ratio is >1.2, a value of +1 is assigned, and when the JNK p54/protein standard ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In the various aspects of this embodiment, when the calculated sum of assigned values is −1 to +1, the results are indeterminate.

In the various aspects of this embodiment, the mRNA standard may be any mRNA standard known and used in the art for similar purposes. Examples include, but are not limited to, L13 mRNA, beta-actin mRNA, glyceraldehyde-3-phosphate dehydrogenase (GAPDH) mRNA, and ribosome small subunit (18S) mRNA. In selected aspects of this embodiment, the mRNA standard is L13 mRNA.

In the various aspects of this embodiment, the protein standard may be any protein standard known and used in the art for similar purposes. Examples include, but are not limited to, β-actin, β-tubulin and GAPDH. In selected aspects of this embodiment, the protein standard is β-actin.

In the various aspects of this embodiment, mRNA expression levels for one, two, three or all of the first biomarkers is determined.

In the various aspects of this embodiment, protein expression levels for one or both of the second biomarkers is determined.

In certain aspects of this embodiment, mRNA expression levels for each of the first biomarkers are determined and protein expression levels for both of the second biomarkers are determined.

In the various aspects of this embodiment, the first and second populations of cells can be the same population of cells (i.e., all analyses are performed on a single population of cells) or different populations of cells. When different populations of cells are used as the first and second populations of cells, the different populations of cells may be from the same source or the different populations of cells may be the same cell type, or both.

As indicated by this embodiment of the invention, the data obtained from the method will indicate whether the subject is undergoing relapse. Based on this information, a physician can determine the best course of new or continuing treatment for the subject. Thus, when the method indicates that the subject is undergoing relapse, the method can further comprise administering a therapeutically effective amount of a treatment for RRMS to the subject. The treatment may be, for example, glatiramer acetate (GA). Other suitable treatments for RRMS include, but are not limited to, beta-interferons, teriflunomide, fingolimod, dimethyl fumarate, natalizumab, ozanimod, ponesimod, ocrelizumab and ofatumumab.

In an exemplary aspect of the first embodiment, the invention is drawn to methods for determining whether a subject having relapsing-remitting multiple sclerosis (RRMS) is undergoing relapse of the disease, comprising
(i) determining mRNA expression levels for one or more first biomarkers selected from the group consisting of SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each selected first biomarker;
(iv) determining protein expression levels for one or more second biomarkers selected from the group consisting of pSIRT1 and JNK p54 in a second population of cells,
(v) determining protein expression levels for β-actin in the same second population of cells, and
(vi) calculating a ratio of second biomarker protein expression to β-actin expression for each selected second biomarker;
wherein when a ratio is calculated for SIRT1 and the SIRT1/L13 mRNA ratio is <3.05, a value of +1 is assigned, and when the SIRT1/L13 mRNA ratio is ≥3.05, a value of −1 is assigned,
wherein when a ratio is calculated for RGC-32 and the RGC-32/L13 mRNA ratio is <1.27, a value of +1 is assigned, and when the RGC-32/L13 mRNA ratio is ≥1.27, a value of −1 is assigned,
wherein when a ratio is calculated for FasL and the FasL/L13 mRNA ratio is <52.6, a value of +1 is assigned, and when the FasL/L13 mRNA ratio is ≥52.6, a value of −1 is assigned,
wherein when a ratio is calculated for IL-21 and the IL-21/L13 mRNA ratio is >16.9, a value of +1 is assigned, and when the IL-21/L13 mRNA ratio is ≤16.9, a value of −1 is assigned,
wherein when a ratio is calculated for pSIRT1 and the pSIRT1/β-actin ratio is <3.05, a value of +1 is assigned, and when the pSIRT1/β-actin ratio is ≥3.05, a value of −1 is assigned,
wherein when a ratio is calculated for JNK p54 and the JNK p54/β-actin ratio is >1.2, a value of +1 is assigned, and when the JNK p54/β-actin ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In a further exemplary aspect of the first embodiment, the invention is drawn to methods for determining whether a subject having relapsing-remitting multiple sclerosis (RRMS) is undergoing relapse of the disease, comprising
(i) determining mRNA expression levels for first biomarkers SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each first biomarker;
(iv) determining protein expression levels for second biomarkers pSIRT1 and JNK p54 in a second population of cells,
(v) determining protein expression levels for a selected protein standard in the same second population of cells, and
(vi) calculating a ratio of second biomarker protein expression to protein standard expression for each second biomarker;
wherein when the ratio calculated for SIRT1 and the SIRT1/mRNA standard ratio is <3.05, a value of +1 is assigned, and when the SIRT1/mRNA standard ratio is ≥3.05, a value of −1 is assigned,
wherein when the ratio calculated for RGC-32 and the RGC-32/mRNA standard ratio is <1.27, a value of +1 is assigned, and when the RGC-32/mRNA standard ratio is ≥1.27, a value of −1 is assigned,
wherein when the ratio calculated for FasL and the FasL/mRNA standard ratio is <52.6, a value of +1 is assigned, and when the FasL/standard mRNA standard ratio is ≥52.6, a value of −1 is assigned,
wherein when the ratio calculated for IL-21 and the IL-21/mRNA standard ratio is >16.9, a value of +1 is assigned, and when the IL-21/mRNA standard ratio is ≤16.9, a value of −1 is assigned,
wherein when the ratio calculated for pSIRT1 and the pSIRT1/protein standard ratio is <3.05, a value of +1 is assigned, and when the pSIRT1/protein standard ratio is ≥3.05, a value of −1 is assigned,
wherein when the ratio calculated for JNK p54 and the JNK p54/protein standard ratio is >1.2, a value of +1 is assigned, and when the JNK p54/protein standard ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In yet a further exemplary aspect of the first embodiment, the invention is drawn to methods for determining whether a subject having relapsing-remitting multiple sclerosis (RRMS) is undergoing relapse of the disease, comprising
(i) determining mRNA expression levels for first biomarkers SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each first biomarker;
(iv) determining protein expression levels for second biomarkers pSIRT1 and JNK p54 in a second population of cells,
(v) determining protein expression levels for β-actin in the same second population of cells, and
(vi) calculating a ratio of second biomarker protein expression to β-actin expression for each second biomarker;
wherein when the ratio calculated for SIRT1 and the SIRT1/L13 mRNA ratio is <3.05, a value of +1 is assigned, and when the SIRT1/L13 mRNA ratio is ≥3.05, a value of −1 is assigned,
wherein when the ratio calculated for RGC-32 and the RGC-32/L13 mRNA ratio is <1.27, a value of +1 is assigned, and when the RGC-32/L13 mRNA ratio is ≥1.27, a value of −1 is assigned,
wherein when the ratio calculated for FasL and the FasL/L13 mRNA ratio is <52.6, a value of +1 is assigned, and when the FasL/L13 mRNA ratio is ≥52.6, a value of −1 is assigned,
wherein when the ratio calculated for IL-21 and the IL-21/L13 mRNA ratio is >16.9, a value of +1 is assigned, and when the IL-21/L13 mRNA ratio is ≤16.9, a value of −1 is assigned,
wherein when the ratio calculated for pSIRT1 and the pSIRT1/β-actin ratio is <3.05, a value of +1 is assigned, and when the pSIRT1/β-actin ratio is ≥3.05, a value of −1 is assigned,
wherein when the ratio calculated for JNK p54 and the JNK p54/β-actin ratio is >1.2, a value of +1 is assigned, and when the JNK p54/β-actin ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

In a second embodiment, the invention is generally drawn to methods for determining whether a subject having relapsing-remitting multiple sclerosis (RRMS) will respond to treatment, such as with glatiramer acetate (GA). In particular, the invention is drawn to methods for determining whether a subject having relapsing-remitting multiple sclerosis (RRMS) will respond to treatment with GA, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <4.33, a value of +1 is assigned, and when the SIRT1/mRNA standard ratio is ≥4.33, a value of −1 is assigned,
wherein when a ratio is calculated for RGC-32 and the RGC-32/mRNA standard ratio is <2.52, a value of +1 is assigned, and when the RGC-32/mRNA standard ratio is ≥2.52, a value of −1 is assigned,
wherein when a ratio is calculated for FasL and the FasL/mRNA standard ratio is <85.4, a value of +1 is assigned, and when the FasL/mRNA standard ratio is ≥85.4, a value of −1 is assigned,
wherein when a ratio is calculated for IL-21 and the IL-21/mRNA standard ratio is >11.9, a value of +1 is assigned, and when the IL-21/mRNA standard ratio is ≤11.9, a value of −1 is assigned,
wherein when a ratio is calculated for pSIRT1 and the pSIRT1/protein standard ratio is <0.3, a value of +1 is assigned, and when the pSIRT1/protein standard ratio is ≥0.3, a value of −1 is assigned,
wherein when a ratio is calculated for JNK p54 and the JNK p54/protein standard ratio is >1.3, a value of +1 is assigned, and when the JNK p54/protein standard ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In the various aspects of this embodiment, when the calculated sum of assigned values is −1 to +1, the results are indeterminate.

In the various aspects of this embodiment, the mRNA standard may be any mRNA standard known and used in the art for similar purposes. Examples include, but are not limited to, L13 mRNA, beta-actin mRNA, glyceraldehyde-3-phosphate dehydrogenase (GAPDH) mRNA, and ribosome small subunit (18S) mRNA. In selected aspects of this embodiment, the mRNA standard is L13 mRNA.

In the various aspects of this embodiment, the protein standard may be any protein standard known and used in the art for similar purposes. Examples include, but are not limited to, β-actin, β-tubulin and GAPDH. In selected aspects of this embodiment, the protein standard is β-actin.

In the various aspects of this embodiment, mRNA expression levels for one, two, three or all of the first biomarkers is determined.

In the various aspects of this embodiment, protein expression levels for one or both of the second biomarkers is determined.

In certain aspects of this embodiment, mRNA expression levels for each of the first biomarkers are determined and protein expression levels for both of the second biomarkers are determined.

In the various aspects of this embodiment, the first and second populations of cells can be the same population of cells (i.e., all analyses are performed on a single population of cells) or different populations of cells. When different populations of cells are used as the first and second populations of cells, the different populations of cells may be from the same source or the different populations of cells may be the same cell type, or both.

As indicated by this embodiment of the invention, the data obtained from the method will indicate whether a subject having relapsing-remitting multiple sclerosis (RRMS) will respond to treatment with glatiramer acetate GA. Based on this information, a physician can determine the best course of new or continuing treatment for the subject. Thus, when it is determined that the subject will respond to treatment with GA, the method may further comprise administering a therapeutically effective amount of GA to the subject. When it is determined that the subject will not respond to treatment with GA, the method may further comprise administering a therapeutically effective amount of a non-GA treatment suitable for RRMS including, but not limited to, beta-interferons, teriflunomide, fingolimod, dimethyl fumarate, natalizumab, ozanimod, ponesimod, ocrelizumab and ofatumumab.

In an exemplary aspect of the second embodiment, the invention is drawn to methods for determining whether a subject having relapsing-remitting multiple sclerosis (RRMS) will respond to treatment with GA, comprising
(i) determining mRNA expression levels for one or more first biomarkers selected from the group consisting of SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each selected first biomarker;
(iv) determining protein expression levels for one or more second biomarkers selected from the group consisting of pSIRT1 and JNK p54 in a second population of cells,
(v) determining protein expression levels for β-actin in the same second population of cells, and
(vi) calculating a ratio of second biomarker protein expression to β-actin expression for each selected second biomarker;
wherein when a ratio is calculated for SIRT1 and the SIRT1/L13 mRNA ratio is <4.33, a value of +1 is assigned, and when the SIRT1/L13 mRNA ratio is ≥4.33, a value of −1 is assigned,
wherein when a ratio is calculated for RGC-32 and the RGC-32/L13 mRNA ratio is <2.52, a value of +1 is assigned, and when the RGC-32/L13 mRNA ratio is ≥2.52, a value of −1 is assigned,
wherein when a ratio is calculated for FasL and the FasL/L13 mRNA ratio is <85.4, a value of +1 is assigned, and when the FasL/L13 mRNA ratio is ≥85.4, a value of −1 is assigned,
wherein when a ratio is calculated for IL-21 and the IL-21/L13 mRNA ratio is >11.9, a value of +1 is assigned, and when the IL-21/L13 mRNA ratio is ≤11.9, a value of −1 is assigned,
wherein when a ratio is calculated for pSIRT1 and the pSIRT1/β-actin ratio is <0.3, a value of +1 is assigned, and when the pSIRT1/β-actin ratio is ≥0.3, a value of −1 is assigned,
wherein when a ratio is calculated for JNK p54 and the JNK p54/β-actin ratio is >1.3, a value of +1 is assigned, and when the JNK p54/β-actin ratio is ≤1.3, a value of −1 is assigned; and
(vii) calculating a sum of the assigned values, wherein when the calculated sum of assigned values is >+1, the subject is determined to respond not to treatment with GA, and when the calculated sum of assigned values is <−1, the subject is determined to respond to treatment with GA.

In a further exemplary aspect of the second embodiment, the invention is drawn to methods for determining whether a subject having relapsing-remitting multiple sclerosis (RRMS) will respond to treatment with GA, comprising
(i) determining mRNA expression levels for first biomarkers SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each first biomarker;
(iv) determining protein expression levels for second biomarkers pSIRT1 and JNK p54 in a second population of cells,
(v) determining protein expression levels for a selected protein standard in the same second population of cells, and
(vi) calculating a ratio of second biomarker protein expression to protein standard expression for each second biomarker;
wherein when the ratio calculated for SIRT1 and the SIRT1/mRNA standard ratio is <4.33, a value of +1 is assigned, and when the SIRT1/mRNA standard ratio is ≥4.33, a value of −1 is assigned,
wherein when the ratio calculated for RGC-32 and the RGC-32/mRNA standard ratio is <2.52, a value of +1 is assigned, and when the RGC-32/mRNA standard ratio is ≥2.52, a value of −1 is assigned,
wherein when the ratio calculated for FasL and the FasL/mRNA standard ratio is <85.4, a value of +1 is assigned, and when the FasL/mRNA standard ratio is ≥85.4, a value of −1 is assigned,
wherein when the ratio calculated for IL-21 and the IL-21/mRNA standard ratio is >11.9, a value of +1 is assigned, and when the IL-21/mRNA standard ratio is ≤11.9, a value of −1 is assigned,
wherein when the ratio calculated for pSIRT1 and the pSIRT1/protein standard ratio is <0.3, a value of +1 is assigned, and when the pSIRT1/protein standard ratio is ≥0.3, a value of −1 is assigned,
wherein when the ratio calculated for JNK p54 and the JNK p54/protein standard ratio is >1.3, a value of +1 is assigned, and when the JNK p54/protein standard ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In yet a further exemplary aspect of the second embodiment, the invention is drawn to methods for determining whether a subject having relapsing-remitting multiple sclerosis (RRMS) will respond to treatment with GA, comprising
(i) determining mRNA expression levels for first biomarkers SIRT1, RGC-32, FasL and IL-21 in a first population of cells,
(ii) determining mRNA expression levels for L13 mRNA in the same first population of cells, and
(iii) calculating a ratio of first biomarker mRNA expression to L13 mRNA expression for each first biomarker;
(iv) determining protein expression levels for second biomarkers pSIRT1 and JNK p54 in a second population of cells,
(v) determining protein expression levels for β-actin in the same second population of cells, and
(vi) calculating a ratio of second biomarker protein expression to β-actin expression for each second biomarker;
wherein when the ratio calculated for SIRT1 and the SIRT1/L13 mRNA ratio is <4.33, a value of +1 is assigned, and when the SIRT1/L13 mRNA ratio is ≥4.33, a value of −1 is assigned,
wherein when the ratio calculated for RGC-32 and the RGC-32/L13 mRNA ratio is <2.52, a value of +1 is assigned, and when the RGC-32/L13 mRNA ratio is ≥2.52, a value of −1 is assigned,
wherein when the ratio calculated for FasL and the FasL/L13 mRNA ratio is <85.4, a value of +1 is assigned, and when the FasL/L13 mRNA ratio is ≥85.4, a value of −1 is assigned,
wherein when the ratio calculated for IL-21 and the IL-21/L13 mRNA ratio is >11.9, a value of +1 is assigned, and when the IL-21/L13 mRNA ratio is ≤11.9, a value of −1 is assigned,
wherein when the ratio calculated for pSIRT1 and the pSIRT1/β-actin ratio is <0.3, a value of +1 is assigned, and when the pSIRT1/β-actin ratio is ≥0.3, a value of −1 is assigned,
wherein when the ratio calculated for JNK p54 and the JNK p54/β-actin ratio is >1.3, a value of +1 is assigned, and when the JNK p54/β-actin ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

In each of the embodiments and aspects of the invention, the methods may include one or more of the following steps: (i) obtaining a biological sample from the subject, such as a sample of cells or a blood sample, (ii) isolating a selected cell type from a biological sample from the subject, (iii) isolating polynucleotides from a selected cell type from a biological sample from the subject, (iv) isolating polypeptides from a selected cell type from a biological sample from the subject, (v) amplifying mRNA from isolated polynucleotides from a selected cell type from a biological sample from the subject, (vi) preparing cDNA from isolated polynucleotides from a selected cell type from a biological sample from the subject, (vii) determining the level of mRNA expression for a selected biomarker in a selected cell type from a biological sample from the subject, (viii) determining the level of cDNA expression for a selected biomarker in a selected cell type from a biological sample from the subject, (ix) determining the level of protein expression for a selected biomarker in a selected cell type from a biological sample from the subject, (x) amplifying L13 mRNA from isolated polynucleotides from a selected cell type from a biological sample from the subject, (xi) amplifying L13 cDNA from isolated polynucleotides from a selected cell type from a biological sample from the subject, (xii) determining the level of L13 mRNA expression in a selected cell type from a biological sample from the subject, (xiii) determining the level of L13 cDNA expression in a selected cell type from a biological sample from the subject, (xiv) determining the level of L13 protein expression in a selected cell type from a biological sample from the subject, and (xv) determining the level of β-actin protein expression in a selected cell type from a biological sample from the subject.

In each of the embodiments and aspects of the invention, the population of cells may be, but is not limited to, peripheral blood mononuclear cells (PBMCs), CD4+ T cells, CD8+ T cells, MAB328+ cells, GFAP+ cells, leukocytes, monocytes, glial cells, and dendritic cells.

In each of the embodiments and aspects of the invention, mRNA expression may be determined using real-time polymerase chain reactions (RT-PCT) or other suitable means.

In each of the embodiments and aspects of the invention, protein expression may be determined using Western blot analysis or other suitable means.

In each of the embodiments and aspects of the invention, the subject is a human, a non-human primate, bird, horse, cow, goat, sheep, a companion animal, such as a dog, cat or rodent, or other mammal.

### III. Examples

### Materials and Methods

### Patients and Controls

The patients were recruited from the University of Maryland Multiple Sclerosis Center. The mean age was 40 (range, 22-60) and the cohort consisted of 60% females (n=9) and 40% males (n=6). The criteria for inclusion of MS patients in the study were: (i) age 18 to 65 years; (ii) fulfillment of the McDonald criteria for definite MS (Polman et al., 2011); (iii) relapsing-remitting course; (iv) having newly diagnosed MS, or MS not treated with currently used immunomodulatory drugs (interferon-(3 or glatiramer acetate) for 3 months prior to study entry; (v) no exacerbations in the 4 weeks before the study; (vi) no i.v. or p.o. steroids for 4 weeks prior to study enrollment; (vii) no treatment with anti-CD20 monoclonal antibodies, natalizumab, fingolimod, mitoxantrone, cyclophosphamide, alemtuzumab, teriflunomide or any investigational drug during the past year; and (viii) a disability score of 0-5.5, as defined by the expanded disability status scale (EDSS) (Kurtzke, 1983). Exclusion criteria for MS patients were: (i) a history of autoimmune disorders, vascular disease, or active acute or chronic infections; (ii) use of antibiotics in the last 30 days; (iii) a history of intracranial or intraspinal tumor or metabolic myelopathy; or (iv) a history of alcohol or drug abuse.

All MS patients received 20 mg of GA injected subcutaneously every day for 2 years. During this 2-year period, patients were clinically evaluated, and peripheral blood samples were collected at 0, 3, 6, and 12 months at the time of their outpatient visits. Patients with symptoms suggestive of a clinical relapse called the University of Maryland Multiple Sclerosis Center. Clinical relapse was defined as substantial worsening of pre-existing symptoms or appearance of new neurological deficits in the absence of fever or infections lasting more than 24 h. An EDSS evaluation was completed at each visit. Clinical records, consultation reports, and inpatient records were reviewed by a neurologist to ensure that the data obtained were complete. In the case of patients with relapse, the administration of 1 g of Solu-Medrol i.v. for 3 days was used to treat the disease exacerbation. A prednisone taper was also used after i.v. Solu-Medrol in certain cases. In such cases, blood samples were obtained prior to Solu-Medrol treatment. Responders to GA treatment were defined as patients who exhibited no relapses during the 2-year span following the initiation of GA. Non-responders were defined as patients who exhibited two or more relapse events during the 2-year span following the initiation of GA. According to these criteria, the present cohort consisted of 11 responders (mean age 43, range 27-60; 55% female) and 4 non-responders (mean age 31, range 22-36; 75% female).

Collection of PBMCs, Total RNA Purification, and cDNA Synthesis

PBMCs were collected using BD Vacutainer CPT tubes (Becton Dickinson, Franklin Lakes, NJ). The mononuclear cells were isolated from fresh blood as previously described (Tegla et al., 2013). RNA isolation was performed the same day, as described earlier (Cudrici et al, 2008). RNA was purified using the RNeasy Mini Kit (Qiagen, Santa Clarita, CA) according to the manufacturer's instructions. RNA (0.5 μg per sample) was mixed with RT buffer, dNTP, and oligo-dT random primers (Invitrogen), then denatured by incubation at 65° C. for 5 min. Reverse transcriptase (Promega) and RNase inhibitor (Invitrogen) were then added, and the reaction mixture was incubated at 37° C. for 1 h to synthesize cDNA. The reaction was terminated by incubating the mixture at 95° C. for 5 min.

### Real-Time PCR

Real-time PCR for RGC-32, FasL, SIRT1, and 11-21 was performed using a StepOne real-time PCR system (Applied Biosystems, Foster City, CA). The primers for the genes investigated were designed and synthesized by IDT (Coralville, IA) (Table 1) and used in conjunction with LightCycler FastStart SYBR Green Master (Roche) along with sample cDNA according to the manufacturer's protocol.

TABLE 1
Primers used for Real-Time PCR

| | | SEQ | |
|---|---|---|---|
| Gene | | ID | Product |
| Symbol | Primer Sequence | NO: | (bp) |
| SIRT1 | For: 5′-TGGCAAAGGAGC | 1 | 159 |
| | AGATTAGTAG-3′ | | |
| | Rev: 5′-GGCATGTCCCAC | 2 | |
| | TATCACTGT-3′ | | |
| HDAC3 | For: 5′-CATGCACCTAGT | 3 | 182 |
| | GTCCAGATTC-3′ | | |
| | Rev: 5′-CACTCTTAAATC | 4 | |
| | TCCACATCGC-3′ | | |
| RGC-32 | For: 5′-AGGAACAGCTTC | 5 | 152 |
| | AGCTTCAG-3′ | | |
| | Rev: 5′-GCTAAAGTTTTG | 6 | |
| | TCAAGATCAGCA-3′ | | |
| FasL | For: 5′-GCCCATTTAACA | 7 | 110 |
| | GGCAAGTC-3′ | | |
| | Rev: 5′-ATCACAAGGCCA | 8 | |
| | CCCTTCTT-3′ | | |
| IL-21 | IL-21 primers were purchased | | |
| | from SABiosciences, | | |
| | cat# PPH01684A (now Qiagen) | | |
| L13 | For: 5′-CGTGCGTCTGAA | 9 | 227 |
| | GCCTACA-3′ | | |
| | Rev: 5′-GGAGTCCGTGGG | 10 | |
| | TCTTGAG-3′ | | |
| CDC2 | For: 5′-TTTTCAGAGCTT | 11 | 100 |
| | TGGGCACT-3′ | | |
| | Rev.: 5′-AGGCTTCCTG | 12 | |
| | GTTTCCATTT-3′ | | |
| AKT1 | For: 5′-ACGCCAAGGAGA | 13 | 185 |
| | TCATGC-3′ | | |
| | Rev.: 5′-CTCCATGCTGT | 14 | |
| | CATCTTGGTC-3′ | | |

Abbreviations: For, forward primer; Rev, reverse primer; bp, base pairs; RGC-32, response gene to complement 32; FasL, Fas ligand; SIRT1, Sirtuin 1; HDAC3, Histone Deacetylase 3; L13, ribosomal protein L13.

As a negative control for each real-time PCR assay, the same reaction was performed in the absence of cDNA. For each gene, the cycle threshold (C T) values were determined in the exponential phase of the amplification plot and normalized to the mRNA expression of L13 ribosomal protein, a housekeeping gene. A standard curve was generated using serial dilutions of qPCR Human Reference Total cDNA (Clontech, Mountain View, CA), and the normalized mRNA value (NRV) was calculated according to the following formula for relative expression of target mRNA: NRV=(TarS/L13), where TarS represents the level of mRNA expression of the target gene, and L13 corresponds to that of the amplified L13 mRNA (Tegla et al., 2013).

### Western Blotting

Western blotting was performed for p-SIRT1 and JNK1 p54 using patient PBMC samples that were lysed in RIPA buffer and processed. Whole-cell lysates (total protein=30-40 μg) were analyzed by 10% SDS-PAGE (BioRad, Hercules, CA), followed by transfer to nitrocellulose membrane and western blotting. Each membrane was analyzed for the expression of 1) p-JNK, using a mouse monoclonal IgG anti p-JNK (G7, Santa Cruz Biotechnology, Dallas, TX); 2) JNK1, using a rabbit polyclonal IgG anti-JNK1 (C-17, Santa Cruz Biotechnology), and 3) p-SIRT1 using rabbit anti-SIRT1 antibodies (Active Motif, Carlsbad, CA). The JNK antibodies recognized both the p46 and p54 isoforms. In the same samples, β-actin (Rockland, Pottstown, PA) was used as a loading control for sample normalization. Anti-rabbit or anti-mouse HRP-conjugated antibody (Santa Cruz Biotechnology) was used as the secondary antibody, and signals were visualized by enhanced chemiluminescence (Denville Scientific, Holliston, MA) and autoradiography. The radiographic band density was measured using UN-SCAN-IT software (Silk Scientific, Orem, UT), and the results were expressed as a ratio to (3-actin as previously described (Tatomir, A. et al., 2018).

### Statistical Analysis

Comparisons between groups were performed using a two-tailed t-test, assuming unequal variances. P values <0.05 were considered significant. Pearson correlation analysis was conducted to examine the association between variables. Statistical analysis was performed using IBM SPSS Statistics software version 22 and GraphPad Prism software version 7. All values are shown as means±SEM and are representative of three experiments unless otherwise noted. Receiver operating characteristic (ROC) curve analysis was used to assess the predictive accuracy of each potential biomarker. The predictive probabilities of binary outcomes regarding clinical state and response to GA treatment were reported as a C-statistic or area under the curve (AUC, represented as a percentage, with a perfect score being 100% predictability).

### MSDA Score

The MSDA score was established by combining the expression of mRNA for Response Gene to Complement-32 (RGC-32), FasL, IL-21, and SIRT1 (as measured by real-time PCR and expressed as a ratio to L13) (Hewes et al. 2017; Kruszewski et al., 2015) with the expression of phosphorylated SIRT1 (pSIRT1) and JNK1 p54 proteins (as measured by Western blotting and expressed as ratios to β-actin) (Anselmo et al., 2020; Ciriello et al., 2018). Four biomarkers (RGC-32, FasL, IL-21, JNK1 p54) were also used.

### Results

### Selection of Cut-Off Values

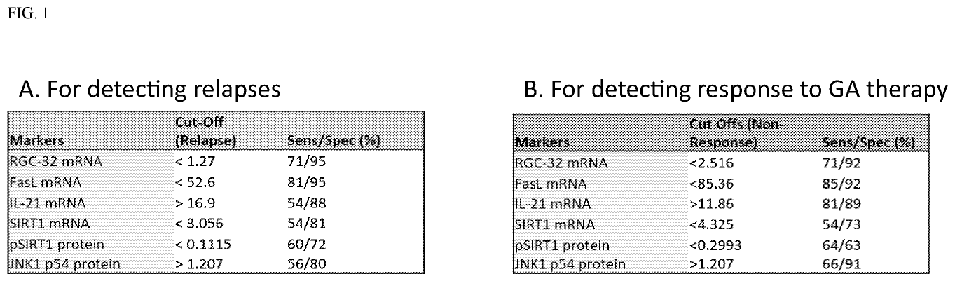

For each biomarker, a cut-off value was generated by using the receiver operating curve (ROC) analysis as previously described (Ciriello et al., 2018; Anselmo et al., 2020; Kruszewski et al., 2015; Hewes et al., 2017). The levels for an individual MS patient were compared with a cut-off value that was previously defined for each of these individual markers (Ciriello et al., 2018; Anselmo et al., 2020; Kruszewski et al., 2015). The biomarkers selected for investigation were derivated from the studies which have shown the major proteins interacting or being regulated by RGC-32 (Ciriello et al., 2018). A cutoff ratio of <1.27 was used for RGC-32, a cut-off level of <52.6 was used for FasL, a cut-off level of >16.9 was used for IL-21, a cut-off level of <3.05 was used for SIRT1, a cut-off of <0.11 was used for p-SIRT1, and a cut-off level of >1.2 was used for JNK1 p54, respectively, for detecting MS disease activity (FIG. 1A).

A RGC-32/L13 ratio <1.27 detected patient relapse with a sensitivity of 71% and a specificity of 95%. A FasL/L13 ratio <52.6 detected patient relapse with a sensitivity of 81% and a specificity of 95%. An IL-21/L13 ratio >16.9 detected patient relapse with a sensitivity of 54% and a specificity of 88% (Kruszewski et al., 2015). A SIRT1/L13 ratio <3.05 detected patient relapse with a sensitivity of 54% and a specificity of 81%. A pSIRT1 cut-off ratio of <0.11 detected relapse with a sensitivity of 60% and a specificity of 72%. A JNK1 p54 cut-off ratio of >1.2 detected relapse with a sensitivity of 56% and a specificity of 80%.

For response to GA treatment cut-off values were: a RGC-32 value of <2.52, a FasL value of <85.4, a IL-21 value of >11.9, a SIRT1 value of <4.33, a p-SIRT1 value of <0.3, and a JNK1 p54 value of >1.3 respectively (FIG. 1B).

### Generation of the MSDA Score

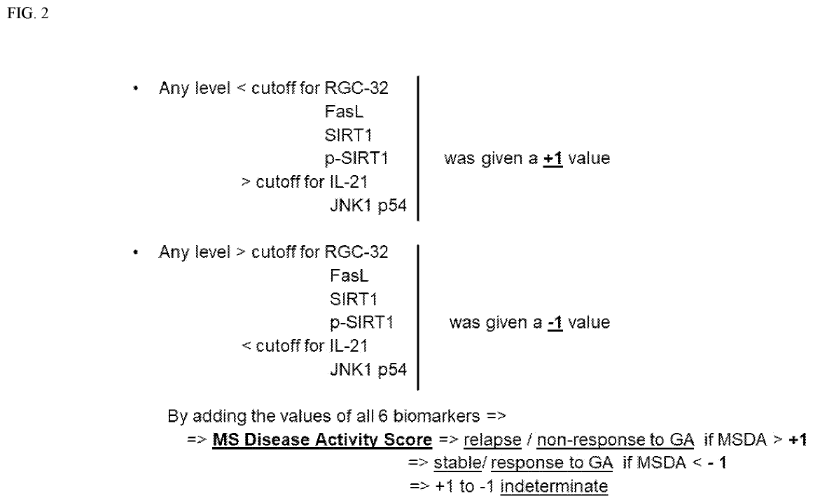

Depending on the levels obtained for each of the six biomarkers examined, a positive or negative value was assigned and used to establish a score for disease activity (FIG. 2). Any value above the cutoff value for RGC-32, FasL, SIRT1, or p-SIRT1 and below the cutoff for IL-21 or JNK1 p54 was considered negative (each biomarker was assigned −1 point) and indicated that the patient was stable or was responding to therapy with GA (FIG. 2). The points given as mentioned above were used to generate a score for disease activity or a score for response to therapy, respectively. A score for disease activity above +1 indicated that the patient has had a relapse or was not responding to treatment. A score below −1 indicated that the patient was stable or was responding well to treatment. A score from +1 to ˜1 is considered indeterminate (FIG. 2).

### Application of MSDA to MS Patients

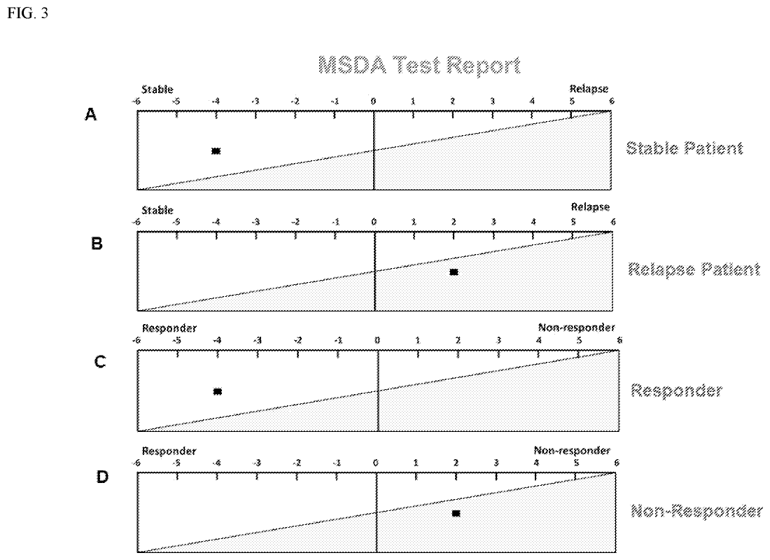

In FIG. 3 examples of patients who were clinically stable (FIG. 3A: MSDA score −4) or had a relapse (FIG. 3B: MSDA score +2) are provided. In addition, an example of a patient who was a responder to treatment with GA (FIG. 3C: MSDA score −4) and a non-responder to GA (FIG. 3D; MSDA score +2) are shown.

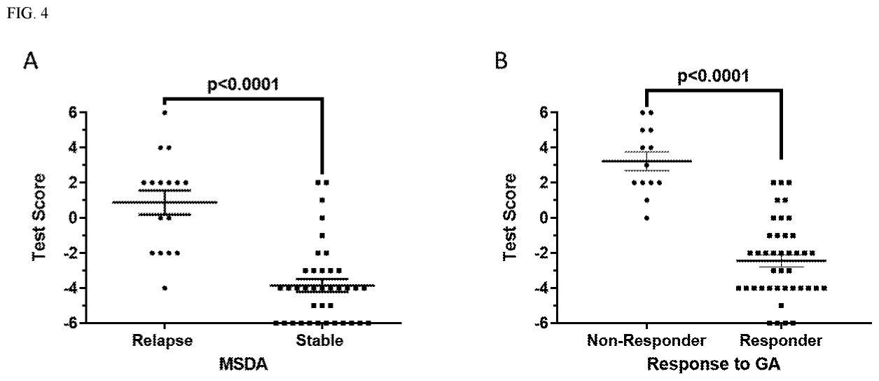

Overall scores for the patients studied are shown in FIG. 4. A statistically significant difference was found between the MSDA scores in stable vs. relapsing patients (p<0.0001) (FIG. 4A) and in non-responders vs. responders (p=0.0001) (FIG. 4B).

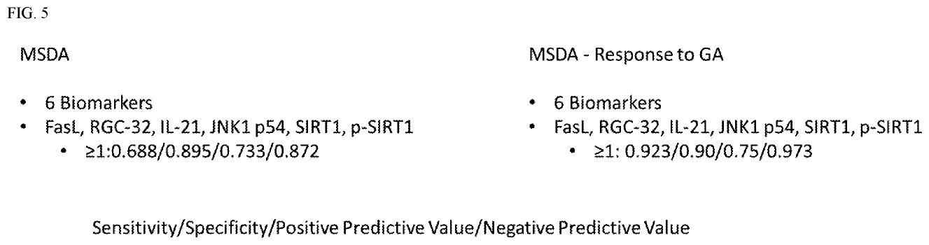

Data on the six biomarkers used in the MSDA score concerning sensitivity, specificity, and predictive value obtained are presented in FIG. 5.

A new multi-biomarker blood test is thus provided for measuring disease activity in patients with MS. This test provided an accurate, reproducible score on a scale from −6 to +6, based on the concentrations of six biomarkers that reflect the pathophysiologic diversity of MS (Platten et al., 2005; Lassmann, 2022). In addition, these markers were previously investigated for their ability to predict relapses and response to GA. The analytical validity, clinical validity, and clinical utility of this test called the MSDA have been evaluated in patients with RRMS. The MSDA has proved to be an easy test to use to identify MS-relapsing vs stable patients. In addition, it can be used to guide treatment with GA in patients with RRMS, and a similar MS response to therapy score can be established for other MS therapies available. As a biomarker-based instrument for assessing disease activity in MS, the MSDA test can help MS physicians monitor the therapeutic response to GA and biologic agents and assess clinically challenging situations, such as when clinical measures are confounded by other inflammatory or non-inflammatory changes from pseudo-relapses (Thrower, 2009). The MSDA scores were positively correlated with EDSS scores, with high MSDA scores being associated with more frequent and severe progression and low scores being predictive of non-progression (R2=0.988, p=0.021).

In summary, the MSDA score is an objective measure of disease activity in MS. By predicting therapeutic response and the risk of relapse, this test has the potential to complement other measures and optimize clinical decision-making. This combination of parameters represents an easily obtained composite biomarker for predicting future disease activity. This test helps identify MS active patients and it is useful in monitoring MS patients with new symptoms. In addition, this multi-biomarker test helps in identifying response to disease modifying therapies in MS by predicting response to treatment.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
Anselmo, F. et al. 2020. JNK and phosphorylated Bcl-2 predict multiple sclerosis clinical activity and glatiramer acetate therapeutic response. Clinical Immunology 210:108297.
Ciriello, J. et al. 2018. Phosphorylated SIRT1 as a biomarker of relapse and response to treatment with glatiramer acetate in multiple sclerosis. Exp Mol Pathol 105:175-180.
Compston, A., Coles, A., 2008. Multiple sclerosis. Lancet. 372, 1502-17.
Cudrici, C. et al., 2008. Complement C5 regulates the expression of insulin-like growth factor binding proteins in chronic experimental allergic encephalomyelitis. J Neuroimmunol 203:94-103.
Frohman, E. M., et al., 2006. Multiple Sclerosis—The Plaque and Its Pathogenesis. New Engl J Med. 354, 942-955.
Grobner, T. 2006. Gadolinium—a specific trigger for the development of nephrogenic fibrosing dermopathy and nephrogenic systemic fibrosis? Nephrol. Dial. Transplant. 21:1104-1108.
Hellman, R. 2011. Gadolinium-Induced Nephrogenic Systemic Fibrosis. Seminars in Nephrology 31:310-316.
Hewes, D. et al. 2017. SIRT1 as a potential biomarker of response to treatment with glatiramer acetate in multiple sclerosis. Exp Mol Pathol 102:191-197.
Keegan, B. M., Noseworthy, J. H., 2002. Multiple sclerosis. Annu Rev Med. 53, 285-302.
Kruszewski A. M. et al. 2015. RGC-32 as a potential biomarker of relapse and response to treatment with glatiramer acetate in multiple sclerosis. Exp Mol Pathol 99:498-505.
Kurtzke, J. F., 1983. Rating neurologic impairment in multiple sclerosis: an expanded disability status scale (EDSS). Neurology. 33, 1444-52.
Lassmann, H., 2022. The contribution of neuropathology to multiple sclerosis research. European Journal of Neurology 29(9):2869-2877.
Lublin, F. D., et al., 2014. Defining the clinical course of multiple sclerosis: the 2013 revisions. Neurology 83:278-86.
Platten, M. and L Steinman, 2005. Multiple sclerosis: trapped in deadly glue. Nat Med 11:252-3.
Polman, C. H., et al., 2011. Diagnostic criteria for multiple sclerosis: 2010 revisions to the McDonald criteria. Ann. Neurol. 69:292-302.
Tatomir, A., et al. 2017. The complement system as a biomarker of disease activity and response to treatment in multiple sclerosis. Immunol Res 65:1103-1109.
Tatomir, A. et al., 2018. RGC-32 regulates reactive astrocytosis and extracellular matrix deposition in experimental autoimmune encephalomyelitis. Immunol Res 66:445-461.
Tegla, C. A., et al., 2013. Dual role of Response gene to complement-32 in multiple sclerosis. Exp Mol Pathol. 94, 17-28.
Thrower, B. W., 2009. Relapse Management in Multiple Sclerosis. The Neurologist 15:1-5.
Trapp, B. D. and K-A Nave. 2008. Multiple Sclerosis: An Immune or Neurodegenerative Disorder? Annual Review of Neuroscience 31:247-269.

What is claimed is:

1. A method for determining whether a subject having relapsing-remitting multiple sclerosis (RRMS) is undergoing relapse of the disease, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <3.05, a value of +1 is assigned, and when the SIRT1/mRNA standard ratio is ≥3.05, a value of −1 is assigned,
wherein when a ratio is calculated for RGC-32 and the RGC-32/mRNA standard ratio is <1.27, a value of +1 is assigned, and when the RGC-32/mRNA standard ratio is ≥1.27, a value of −1 is assigned,
wherein when a ratio is calculated for FasL and the FasL/mRNA standard ratio is <52.6, a value of +1 is assigned, and when the FasL/mRNA standard ratio is ≥52.6, a value of −1 is assigned,
wherein when a ratio is calculated for IL-21 and the IL-21/mRNA standard ratio is >16.9, a value of +1 is assigned, and when the IL-21/mRNA standard ratio is ≤16.9, a value of −1 is assigned,
wherein when a ratio is calculated for pSIRT1 and the pSIRT1/protein standard ratio is <3.05, a value of +1 is assigned, and when the pSIRT1/protein standard ratio is ≥3.05, a value of −1 is assigned,
wherein when a ratio is calculated for JNK p54 and the JNK p54/protein standard ratio is >1.2, a value of +1 is assigned, and when the JNK p54/protein standard ratio is ≤1.2, a value of −1 is assigned; and
(vii) calculating a sum of the assigned values, wherein when the calculated sum of assigned values is >+1, the subject is determined to be undergoing relapse, and when the calculated sum of assigned values is <−1, the subject is determined not to be undergoing relapse.

2. The method of claim 1, wherein the mRNA standard is selected from the group consisting of L13 mRNA, beta-actin mRNA, glyceraldehyde-3-phosphate dehydrogenase (GAPDH) mRNA, and ribosome small subunit (18S) mRNA.

3. The method of claim 1, wherein the protein standard is selected from the group consisting of β-actin, β-tubulin and GAPDH.

4. The method of claim 1, wherein mRNA expression levels for each of the first biomarkers are determined and protein expression levels for both of the second biomarkers are determined.

5. The method of claim 1, further comprising administering a therapeutically-effective amount of a treatment for RRMS to the subject when the subject is determined to be undergoing relapse.

6. The method of claim 5, wherein the treatment is selected from the group consisting of glatiramer acetate (GA), beta-interferons, teriflunomide, fingolimod, dimethyl fumarate, natalizumab, ozanimod, ponesimod, ocrelizumab and ofatumumab.

7. The method of claim 1, wherein the population of cells is selected from the group consisting of peripheral blood mononuclear cells (PBMCs), CD4+ T cells, CD8+ T cells, MAB328+ cells, GAFP+ cells, leukocytes, monocytes, glial cells, and dendritic cells.

8. The method of claim 1, wherein mRNA expression is determined using real-time polymerase chain reaction (RT-PCT).

9. The method of claim 1, wherein protein expression is determined using Western blot analysis.

10. A method for determining whether a subject having relapsing-remitting multiple sclerosis (RRMS) will respond to treatment with GA, comprising
(i) determining mRNA expression levels for one or more first biomarkers selected from the group consisting of SIRT1, RGC-32, FasL and IL-21 in a first population of cells,
(ii) determining mRNA expression levels for a selected mRNA standard in the same first population of cells, and
(iii) calculating a ratio of first biomarker mRNA expression to mRNA standard expression for each selected first biomarker;
(iv) determining protein expression levels for one or more second biomarkers selected from the group consisting of pSIRT1 and JNK p54 in a second population of cells,
(v) determining protein expression levels for a selected protein standard in the same second population of cells, and
(vi) calculating a ratio of second biomarker protein expression to protein standard expression for each selected second biomarker;
wherein when a ratio is calculated for SIRT1 and the SIRT1/mRNA standard ratio is <4.33, a value of +1 is assigned, and when the SIRT1/mRNA standard ratio is ≥4.33, a value of −1 is assigned,
wherein when a ratio is calculated for RGC-32 and the RGC-32/mRNA standard ratio is <2.52, a value of +1 is assigned, and when the RGC-32/mRNA standard ratio is ≥2.52, a value of −1 is assigned,
wherein when a ratio is calculated for FasL and the FasL/mRNA standard ratio is <85.4, a value of +1 is assigned, and when the FasL/mRNA standard ratio is ≥85.4, a value of −1 is assigned,
wherein when a ratio is calculated for IL-21 and the IL-21/mRNA standard ratio is >11.9, a value of +1 is assigned, and when the IL-21/mRNA standard ratio is ≤11.9, a value of −1 is assigned,
wherein when a ratio is calculated for pSIRT1 and the pSIRT1/protein standard ratio is <0.3, a value of +1 is assigned, and when the pSIRT1/protein standard ratio is ≥0.3, a value of −1 is assigned,
wherein when a ratio is calculated for JNK p54 and the JNK p54/protein standard ratio is >1.3, a value of +1 is assigned, and when the JNK p54/protein standard ratio is ≤1.3, a value of −1 is assigned; and
(vii) calculating a sum of the assigned values, wherein when the calculated sum of assigned values is >+1, the subject is determined not to respond to treatment with GA, and when the calculated sum of assigned values is <−1, the subject is determined to respond to treatment with GA.

11. The method of claim 10, wherein the mRNA standard is selected from the group consisting of L13 mRNA, beta-actin mRNA, glyceraldehyde-3-phosphate dehydrogenase (GAPDH) mRNA, and ribosome small subunit (18S) mRNA.

12. The method of claim 10, wherein the protein standard is selected from the group consisting of β-actin, β-tubulin and GAPDH.

13. The method of claim 10, wherein mRNA expression levels for each of the first biomarkers are determined and protein expression levels for both of the second biomarkers are determined.

14. The method of claim 10, further comprising administering a therapeutically-effective amount of GA to the subject when the subject is determined to respond to treatment with GA.

15. The method of claim 10, further comprising administering a therapeutically-effective amount of a non-GA treatment to the subject when the subject is determined not to respond to treatment with GA.

16. The method of claim 15, wherein the non-GA treatment is selected from the group consisting of glatiramer acetate (GA), beta-interferons, teriflunomide, fingolimod, dimethyl fumarate, natalizumab, ozanimod, ponesimod, ocrelizumab and ofatumumab.

17. The method of claim 10, wherein the population of cells is selected from the group consisting of peripheral blood mononuclear cells (PBMCs), CD4+ T cells, CD8+ T cells, MAB328+ cells, GFAP+ cells, leukocytes, monocytes, glial cells, and dendritic cells.

18. The method of claim 10, wherein mRNA expression is determined using real-time polymerase chain reaction (RT-PCT).

19. The method of claim 10, wherein protein expression is determined using Western blot analysis.


SEQUENCE LISTING (14 sequences)

SEQ ID NO: 1 - DNA, 22 bp, synthetic construct
  misc_feature 1..22: note = PCR primer
  source 1..22: mol_type = other DNA
tggcaaaggagcagattagtag

SEQ ID NO: 2 - DNA, 21 bp, synthetic construct
  misc_feature 1..21: note = PCR primer
  source 1..21: mol_type = other DNA
ggcatgtcccactatcactgt

SEQ ID NO: 3 - DNA, 22 bp, synthetic construct
  misc_feature 1..22: note = PCR primer
  source 1..22: mol_type = other DNA
catgcacctagtgtccagattc

SEQ ID NO: 4 - DNA, 22 bp, synthetic construct
  misc_feature 1..22: note = PCRF primer
  source 1..22: mol_type = other DNA
cactcttaaatctccacatcgc

SEQ ID NO: 5 - DNA, 20 bp, synthetic construct
  misc_feature 1..20: note = PCR primer
  source 1..20: mol_type = other DNA
aggaacagcttcagcttcag

SEQ ID NO: 6 - DNA, 24 bp, synthetic construct
  misc_feature 1..24: note = PCR primer
  source 1..24: mol_type = other DNA
gctaaagttttgtcaagatcagca

SEQ ID NO: 7 - DNA, 20 bp, synthetic construct
  misc_feature 1..20: note = PCR primer
  source 1..20: mol_type = other DNA
gcccatttaacaggcaagtc

SEQ ID NO: 8 - DNA, 20 bp, synthetic construct
  misc_feature 1..20: note = PCR primer
  source 1..20: mol_type = other DNA
atcacaaggccacccttctt

SEQ ID NO: 9 - DNA, 19 bp, synthetic construct
  misc_feature 1..19: note = PCR primer
  source 1..19: mol_type = other DNA
cgtgcgtctgaagcctaca

SEQ ID NO: 10 - DNA, 19 bp, synthetic construct
  misc_feature 1..19: note = PCR primer
  source 1..19: mol_type = other DNA
ggagtccgtgggtcttgag

SEQ ID NO: 11 - DNA, 20 bp, synthetic construct
  misc_feature 1..20: note = PCR primer
  source 1..20: mol_type = other DNA
ttttcagagctttgggcact

SEQ ID NO: 12 - DNA, 20 bp, synthetic construct
  misc_feature 1..20: note = PCR primer
  source 1..20: mol_type = other DNA
aggcttcctggtttccattt

SEQ ID NO: 13 - DNA, 18 bp, synthetic construct
  misc_feature 1..18: note = PCR primer
  source 1..18: mol_type = other DNA
acgccaaggagatcatgc

SEQ ID NO: 14 - DNA, 21 bp, synthetic construct
  misc_feature 1..21: note = PCR primer
  source 1..21: mol_type = other DNA
ctccatgctgtcatcttggtc